(19) United States
(12) Patent Application Publication
(10) Pub. No.: US 2025/0122236 A1
(43) Pub. Date: Apr. 17, 2025

(54) INTERMEDIATE FOR PREPARING FULVESTRANT AND PREPARATION METHOD THEREFOR

(71) Applicant: SHANGHAI BEST-LINK BIOSCIENCE, LLC, Pudong, Shanghai (CN)

(72) Inventors: Fuyao ZHANG, Pudong, Shanghai (CN); Xianjie CHEN, Pudong, Shanghai (CN)

(21) Appl. No.: 18/568,498

(22) PCT Filed: Jun. 6, 2022
(86) PCT No.: PCT/CN2022/097214

§ 371 Date: Dec. 8, 2023

(30) Foreign Application Priority Data
Jun. 9, 2021 (CN) 202110644317.9

(51) Int. Cl. C07J 31/00; C07F 3/02; C07J 1/00

(52) U.S. Cl. CPC C07J 31/006; C07F 3/02; C07J 1/0074

(57) ABSTRACT

The invention relates to a fulvestrant intermediate and a preparation method thereof. Specifically, the invention relates to a compound shown as a formula V and a preparation method thereof, and R¹ is hydrogen or a hydroxyl protecting group. The invention also relates to a preparation method for the synthesis of fulvestrant from the compound as shown in the formula V. The method has the advantages of short reaction steps, mild and safe reaction conditions, high selectivity, simplicity and convenience in operation and purification, high synthesis efficiency and the like, and is suitable for large-scale production.

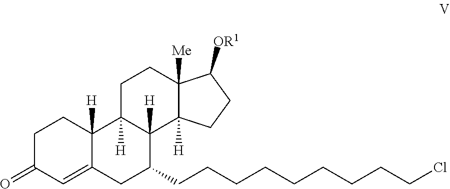

This application claims priority to Chinese Application No 2021106443179, filed on Jun. 9, 2021 The entire disclosures of all of the above applications are incorporated herein by reference.

### FIELD OF THE INVENTION

The invention relates to a fulvestrant intermediate and a preparation method thereof.

### BACKGROUND OF THE INVENTION

Fulvestrant (formula I) is an estrogen receptor antagonist. In April 2002, AstraZeneca's fulvestrant injection was approved by the FDA, and its global sales exceeded $1 billion in 2018.

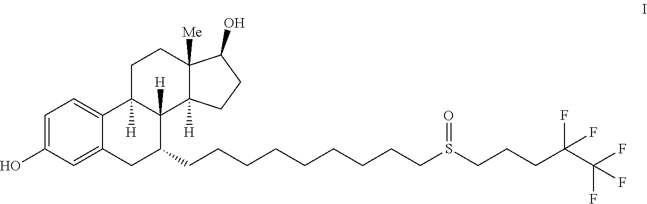

The current synthesis of fulvestrant is mainly carried out by introducing the side chain at C7 position of the steroid, roughly through two strategies, alkylation and addition of Grignard reagent.

As disclosed in the patents CN102827048, CN102993257, CN110938107, CN102993259, WO2009013310, WO2009039700, U.S. Pat. No. 9,315,540, the alkylation route uses estradiol as starting material. After functional group protection, oxidation at C6 position is required to form alcohol, which is further oxidized to ketone. After the alkylation reaction, the ketone needs to be reduced to obtain the parent structure of fulvestrant. The reaction operations are cumbersome.

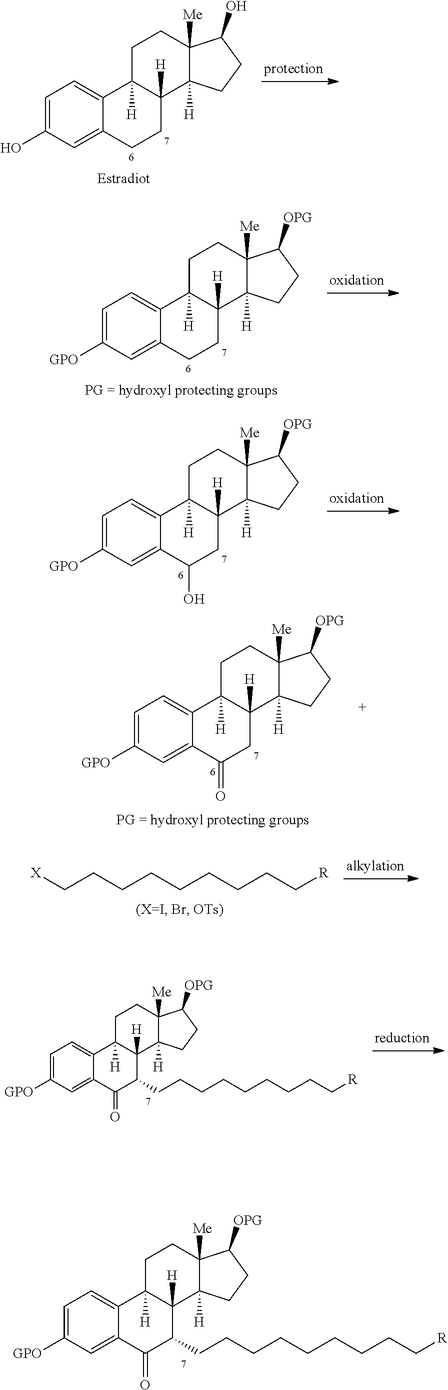

The Grignard addition route uses dehydronandrolone acetate as starting material, and the parent structure of fulvestrant was obtained in two steps of Grignard addition and aromatization. As for structures of Grignard reagents, patents WO2002032922 and WO2006015081 disclose the Grignard addition route when R is

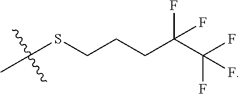 [SMILES: *SCCCC(F)(F)C(F)(F)F]

Although this route is very simple, it is mentioned in its corresponding literature Organic Process Research & Development 2010, 14, 544-552 that when the structure contains both sulfur and bromine, the bromine containing precursor of Grignard reagent is unstable and easy to decompose. The presence of small amount of impurities would also lead to difficulties in the initiation of Grignard reagent, so the requirements for the reservation and purity of the materials are very high

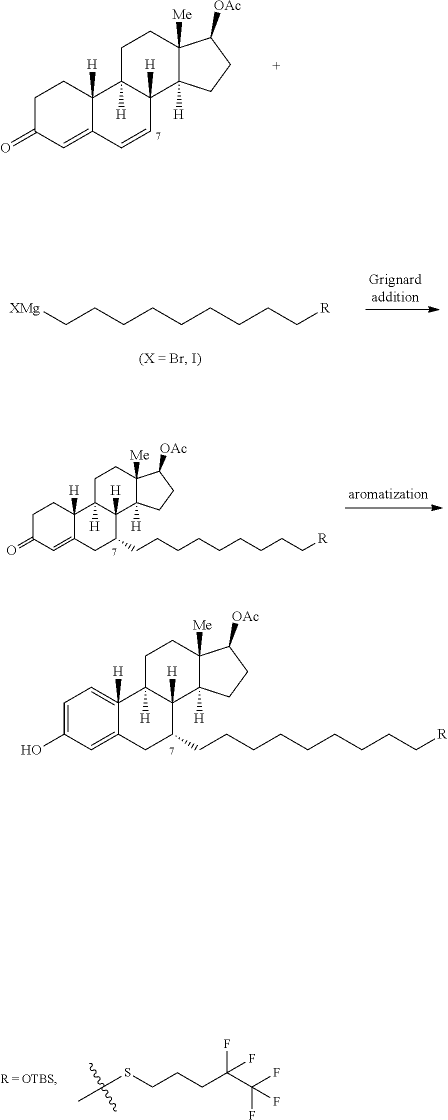

Patents WO2006015081 and CN103965280B also disclose the protocol for the addition of dehydronandrolone acetate by Grignard reagents with relatively shorter chain when R is OTBS. However, removal of TBS protection group and conversion of hydroxyl group to a leaving group are required before the introduction of pentafluoropentanethiol fragment, which increases steps to the synthesis route and difficulties of purification.

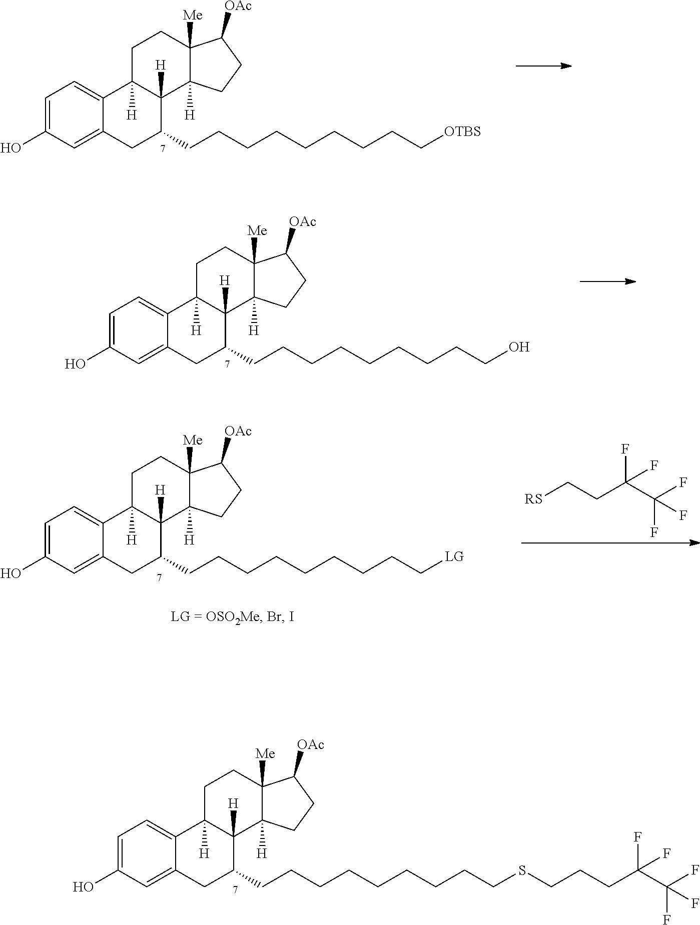

In summary, the existing methods for the preparation of fulvestrant require repeated oxidation/reduction reactions on the skeleton structure, or protection and deprotection reactions on the side chain, resulting in cumbersome synthetic routes, difficult purification, and low efficiency. Therefore, a more efficient synthetic route to prepare fulvestrant needs to be developed.

### CONTENT OF THE PRESENT INVENTION

To overcome the draw backs of the existing preparation method of fulvestrant, the inventor made great efforts to invent a new intermediate and synthesis method for the preparation of fulvestrant, and the preparation method for the synthesis of fulvestrant from the intermediate. This method has a short synthesis route, with mild and safe reaction conditions, simple operation and purification, high selectivity, high efficiency, and is suitable for large-scale production of fulvestrant.

The present invention provides compounds represented by general formula V:

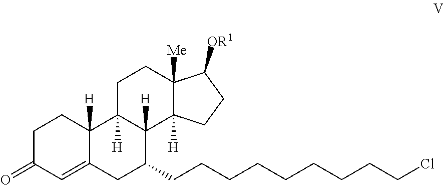

wherein:
R¹ is hydrogen or hydroxyl protecting groups.

In a preferred embodiment of the present invention, in the compound shown in formula V, R¹ is hydrogen, C₁₋₆ alkyl, C₁₋₁₀ alkyl acyl, aryl acyl or (C₁₋₁₀ alkyl or aryl), silyl.

In a more preferred embodiment of the present invention, in the compound shown in formula V, R¹ is C₁₋₁₀ alkyl acyl or aryl acyl.

In a more preferred embodiment of the present invention, in the compound shown in formula V, R¹ is acetyl.

The present invention also provides a method for preparing compounds shown in formula V, which comprises the following step the addition reaction of compounds shown in formula VII to compounds shown in formula VI gives compounds shown in formula V; The addition reaction is preferably carried out under the catalysis of copper reagents:

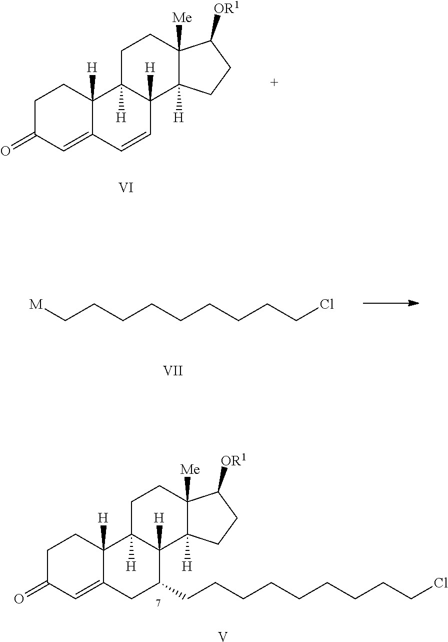

wherein:
R¹ is hydrogen or hydroxyl protecting groups, M is MgCl, MgBr, MgI, Mg_{1/2}, ZnCl, ZnBr, ZnI, Zn_{1/2}, MnCl, Li or Na.

In a preferred embodiment of the present invention, in the preparation method of the compound shown in formula V, R¹ is hydrogen, C₁₋₆ alkyl, C₁₋₁₀ alkyl acyl, aryl acyl or (C₁₋₁₀ alkyl or aryl)₃ silyl, M is MgCl or MgBr.

In a more preferred embodiment of the present invention, in the preparation method of compound shown in formula V, R¹ is C₁₋₁₀ alkyl acyl or aryl acyl group; M is MgCl or MgBr.

In a more preferred embodiment of the present invention, in the preparation method of compound shown in formula V, R¹ is acetyl; M is MgCl or MgBr.

In the preparation method of the compound shown in formula V, the addition reaction produces a set of diastereomer mixtures. As an example, the ratio of the diastereomer V-7α, V-7β can be 100˜1:1. The diastereomer mixture can be directly subjected to the next reaction, or it can be purified to obtain a single diastereomer and then proceed to the next reaction

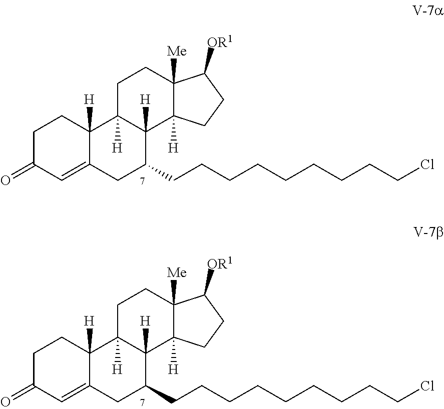

In the preparation method of the compound shown in formula V, the addition reaction can be carried out in organic solvents. The organic solvent can be a conventional solvent for this type of reaction in this field, and can also be one or more of C₆₋₁₀ alkane solvents (e.g., n-hexane, n-heptane), aromatic solvents (e.g., toluene), ether solvents (e.g., tetrahydrofuran, 2-methyltetrahydrofuran, methyl tert-butyl ether, dioxane). The amount of organic solvent can be a conventional amount of this type of reaction in this field, for example, the molar concentration of the compound shown in formula VI in the organic solvent can be 0.001˜5 mol/L.

In the preparation method of the compound shown in formula V, the more preferred molar concentration of the compound shown in formula VI in the organic solvent is 0.005, 0.01, 0.05, 0.1, 0.5, 1.0 or 5.0 mol/L.

In the preparation method of the compound shown in formula V, the copper reagents can be one or more of CuCl, CuBr, CuI, CuOTf. The amount of copper reagent can be a conventional amount of this type of reaction in this field, for example, the molar ratio of the copper reagent to the compound shown in formula VI can be 0.001˜1:1.

In the preparation method of the compound shown in formula V, the more preferred molar ratio of the copper reagent to the compound shown in formula VI is 0.01:1, 0.05:1, 0.1:1, 0.3:1, 0.5:1 or 1:1.

In the preparation method of the compound shown in formula V, the molar ratio of the compound shown in formula VII to the compound shown in formula VI can be 1˜10:1.

In the preparation method of the compound shown in formula V, the more preferred molar ratio of the compound shown in formula VII to the compound shown in formula VI is 5:1, 4:1, 3:1, 2:1 or 1:1.

In the preparation method of the compound shown in formula V, the progress of the addition reaction can be monitored by conventional methods in this field (such as TLC, HPLC, GC or NMR, preferably HPLC), generally when the compound shown in formula VI no longer reacts as the reaction endpoint.

In the preparation method of the compound shown in formula V, the reaction temperature of the addition reaction can be conventional temperature of this type of reaction in this field, for example, −50˜50° C.

In the preparation method of the compound shown in formula V, the more preferred reaction temperature of the addition reaction is −30° C., −20° C., −10° C., 0° C. 10° C. or 20° C.

The present invention also provides a compound as shown in formula VII,

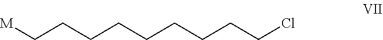

wherein:
M is MgCl, MgBr, MgI, Mg_{1/2}, ZnCl, ZnBr, ZnI, Zn_{1/2}, MnCl, Li or Na.

In a preferred embodiment of the present invention. M is MgCl or MgBr.

In a more preferred embodiment of the present invention. M is MgBr.

The present invention also provides a method for preparing a compound as shown in formula VII, which comprises the following step: the compound as described in formula VIII reacts with metal, or exchanges with organometallic compound to prepare a compound as shown in formula VII,

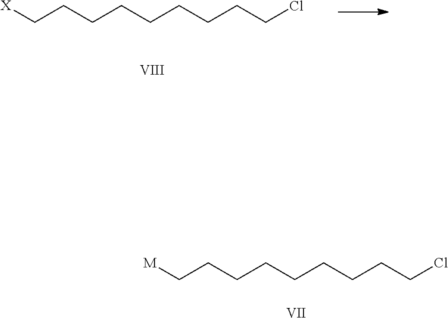

wherein:
X is Cl, Br or I; N is MgCl, MgBr, MgI, Mg_{1/2}, ZnCl, ZnBr, ZnI, Zn_{1/2}, MnCl, Li or Na.

In a preferred embodiment of the present invention, the preparation method of the compound shown in formula VII, X is Cl or Br, M is MgCl or MgBr.

In a more preferred embodiment of the present invention, the preparation method of the compound shown in formula VII, X is Br, M is MgBr.

In a preferred embodiment of the present invention, the preparation method of the compound shown in formula VII, the compound in formula VIII reacts with magnesium metal to prepare the compound shown in formula VII, the magnesium metal can be one or more of magnesium powder, magnesium turning.

In the preparation method of the compound shown in formula VII, the Grignard reaction can be carried out in organic solvents. The organic solvent can be a conventional solvent for this type of reaction in this field, for example, one or more of ether solvent (e.g., tetrahydrofuran, 2-methyltetrahydrofuran, methyl tert-butyl ether, ether, dioxane). The amount of organic solvent can be a conventional amount of this type of reaction in this field, for example, the molar concentration of the compound shown in formula VIII in the organic solvent can be 0.001˜5 mol/L.

In the preparation method of the compound shown in formula VII, the more preferred molar concentration of the compound shown in formula VIII in the organic solvent is 0.005, 0.01, 0.05, 0.1, 0.5, 1.0 or 5.0 mol/L.

In the preparation method of the compound shown in formula VII, the molar ratio of the magnesium metal to the compound shown in formula VIII can be 0.5˜0.1.

In the preparation method of the compound shown in formula VII, the more preferred molar ratio of the metal magnesium to the compound shown in formula VIII is 0.5:1, 0.8:1, 0.9:1, 0.95:1, 1:1, 1.5:1, 2:1, 3:1 or 5:1.

In the preparation method of the compound shown in formula VII, the reaction temperature of the Grignard reaction can be conventional temperature of this type of reaction in this field, for example, −20˜80° C.

In the preparation method of the compound shown in formula VII, the more preferred reaction temperature of the Grignard reaction is −10° C., 0° C., 10° C., 20° C., 30° C., 40° C., 50° C. or 80° C.

The present invention also provides a compound as shown in formula III,

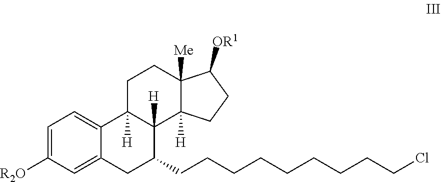

wherein:
R¹ is hydrogen or hydroxyl protecting groups.

In a preferred embodiment of the present invention, in the compound shown in formula III, R¹ is hydrogen, C₁₋₆ alkyl, C₁₋₁₀ alkyl acyl, aryl acyl or (C₁₋₁₀ alkyl or aryl)₃ silyl; R¹ is C₁₋₁₀ alkyl acyl, aryl acyl.

In a more preferred embodiment of the present invention, in the compound shown in formula III, R¹ is C₁₋₁₀ alkyl acyl or aryl acyl; R² is C₁₋₁₀ alkyl acyl or aryl acyl.

In a more preferred embodiment of the present invention, in the compound shown in formula III, R¹ is acetyl; R² is acetyl or propionyl.

The present invention also provides a method for preparing compounds as shown in formula III, which comprises the following steps: the compound shown in formula V undergoes aromatization reaction and phenolic hydroxyl protection reaction to a compound as indicated in formula III; the aromatization reaction is preferably carried out with copper salts:

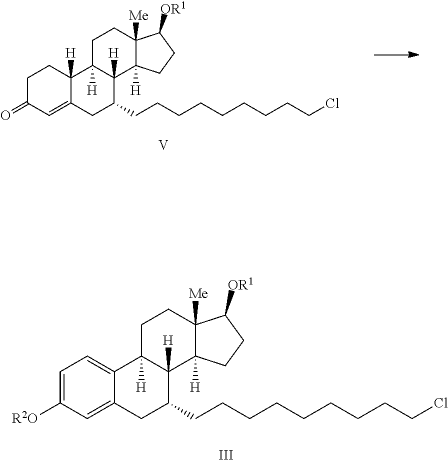

wherein:
R¹ is hydrogen or hydroxyl protecting groups, R² is hydroxyl protecting groups.

In a preferred embodiment of the present invention, in the preparation method of the compound shown in formula III, R¹ is hydrogen, C₁₋₆ alkyl, C₁₋₁₀ alkyl acyl, aryl acyl or (C₁₋₁₀ alkyl or aryl)₃ silyl; R² is C₁₋₁₀ alkyl acyl or aryl acyl.

In a more preferred embodiment of the present invention, in the preparation method of the compound shown in formula III, R¹ is C₁₋₁₀ alkyl acyl or aryl acyl; R² is C₁₋₁₀ alkyl acyl or aryl acyl.

In a more preferred embodiment of the present invention, in the preparation method of the compound shown in formula III, R¹ is acetyl; R² is acetyl or propionyl.

In a more preferred embodiment of the present invention, in the preparation method of the compound shown in formula III, R¹ is acetyl; R² is acetyl.

In the preparation method of the compound shown in formula III, the aromatization reaction can be carried out in organic solvents. The organic solvent can be a conventional solvent for this type of reaction in this field, and can also be one or more of nitrile solvent (e.g., acetonitrile, propionitrile, butyronitrile, benzonitrile), amide solvent (e.g., N,N-dimethylformamide, N,N-dimethylacetamide), sulfone solvent (e.g., dimethyl sulfoxide, tetramethylene sulfone), aromatic solvents (e.g., toluene), ether solvents (e.g., tetrahydrofuran, 2-methyltetrahydrofuran, methyl tert-butyl ether, dioxane). The amount of organic solvent can be a conventional amount of this type of reaction in this field, for example, the molar concentration of the compound shown in formula V in the organic solvent can be 0.01˜0.5 mol/L.

In the preparation method of the compound shown in formula III, the more preferred molar concentration of the compound shown in formula V in the organic solvent is 0.005, 0.01, 0.05, 0.1, 0.5, 1.0 or 5.0 mol/L.

In the preparation method of the compound shown in formula III, the copper salt can be one or more of CuCl₂, CuBr₂, CuI, CuSO₄, Cu(OAc)₂, Cu(NO₃)₂, Cu(CF₃SO₃)₂. The amount of copper salt can be a conventional amount of this type of reaction in this field, for example, the molar ratio of the copper salt to the compound shown in formula V can be 0.5˜10.1.

In the preparation method of the compound shown in formula III, the more preferred molar ratio of the copper salt to the compound shown in formula V is 0.5:1, 0.8:1, 0.9:1, 1.1, 2:1, 3:1 or 5:1.

In the preparation method of the compound shown in formula III, the phenolic hydroxyl protection reaction proceeds with acylation reagent. The acylation reagent can be one or more of the alkyl acylation reagents (e.g., acetic anhydride, acetyl chloride, propionic anhydride, propionyl chloride) or aryl acylation reagents (e.g., benzoyl chloride, benzoic anhydride, p-toluoyl chloride, p-toluic anhydride). The amount of acylation reagent can be a conventional amount of this type of reaction in this field, for example, the molar ratio of the acylation reagent to the compound shown in formula V can be 1˜10:1.

In the preparation method of the compound shown in formula III, the amount of acylation reagent can be a conventional amount of this type of reaction in this field, for example, the more preferred molar ratio of the acylation reagent to the compound shown in formula V is 1:1, 2:1, 3:1, 5:1 or 8:1.

In the preparation method of the compound shown in formula III, the progress of the aromatization reaction can be monitored by conventional methods in this field (such as TLC, HPLC, GC or NMR, preferably HPLC), generally when the compound shown in formula V no longer reacts as the reaction endpoint.

In the preparation method of the compound shown in formula III, the reaction temperature of the aromatization reaction can be conventional temperature of this type of reaction in this field, for example, −20˜60° C.

In the preparation method of the compound shown in formula III, the more preferred reaction temperature of the aromatization reaction is −10° C., 0° C., 10° C., 20° C., 30° C., 40° C., 50° C. or 60° C.

The present invention also provides a method for preparing fulvestrant as shown in formula I, specifically, comprising the following steps,

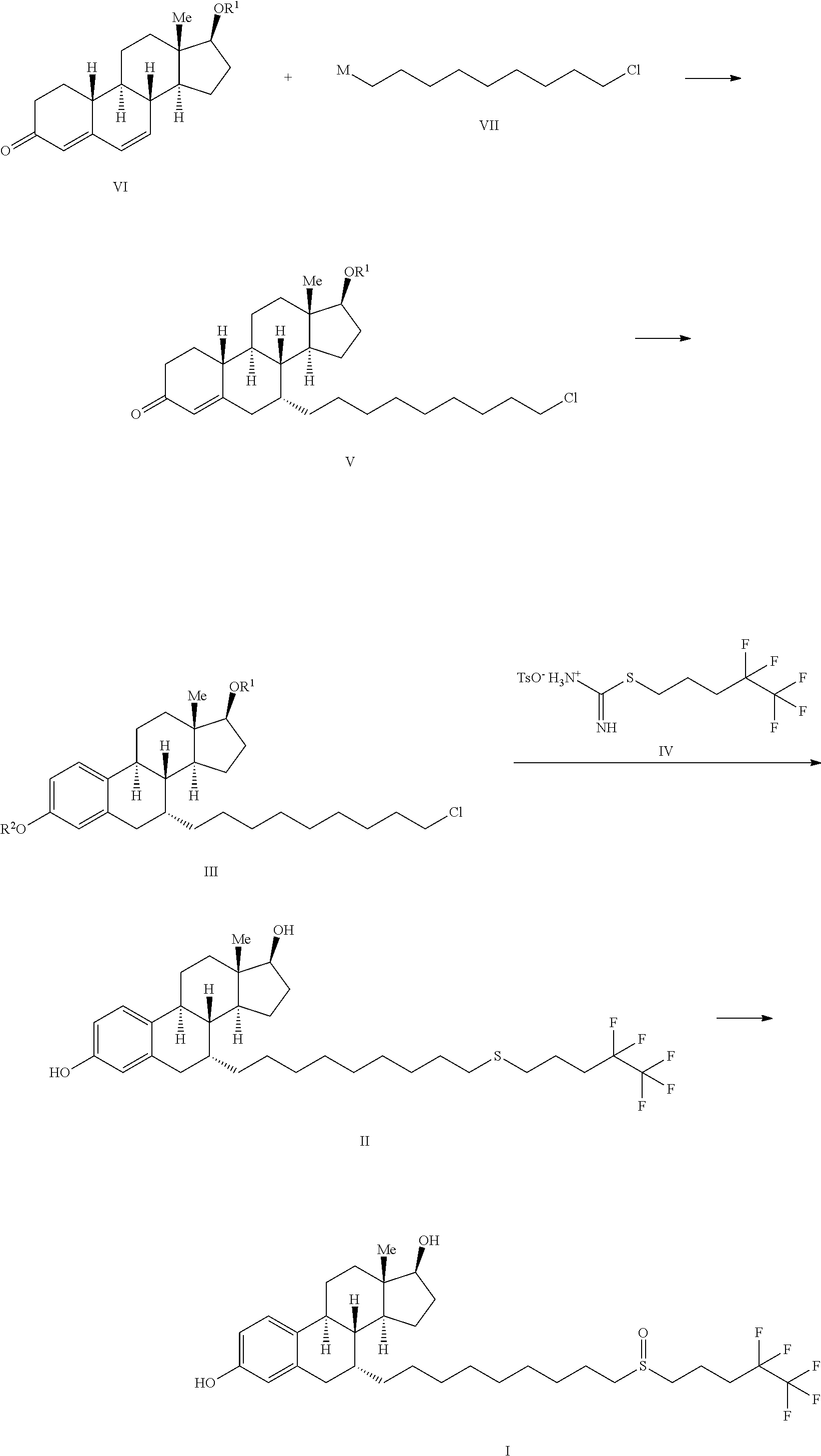

wherein:
R¹ is hydrogen or hydroxyl protecting groups; R² is hydroxyl protecting groups; M is MgCl, MgBr, MgI, Mg_{1/2}, ZnCl, ZnBr, ZnI, Zn_{1/2}, MnCl, Li or Na.
1) The compound shown in formula VI and the compound shown in formula VII undergo addition reactions to obtain the compound shown in formula V; The addition reaction is preferably carried out under the action of a copper reagent; The preferred copper reagent is one or more of CuCl, CuBr, CuI, CuOTf;
2) The compound shown in formula V undergoes aromatization reaction and phenolic hydroxyl protection reaction to obtain the compound shown in formula III; The aromatization reaction is preferably carried out under the action of copper salts; The preferred copper salt is one or more of CuCl₂, CuBr, CuI, CuSO₄, Cu(OAc)₂, Cu(NO₃)₂, Cu(CF₃SO₃)₂; The phenolic hydroxyl protection reaction is completed under the action of acylation reagent, and the acylation reagent is acetic anhydride or propionic anhydride;
3) The compound shown in formula III and the compound shown in formula IV undergo substitution reactions to obtain the compound as shown in formula II; The substitution reaction is preferably carried out under the action of base; The preferred base is one or more of sodium hydroxide, potassium hydroxide, lithium hydroxide, sodium carbonate, potassium carbonate, cesium carbonate;
4) The compounds shown in formula II undergo oxidation reactions to obtain the compounds shown in formula I; The oxidation reaction is preferably carried out under the action of an oxidant; The oxidant is preferably one or more of hydrogen peroxide, m-chloroperoxybenzoic acid, tert-butyl hydroperoxide, peracetic acid:

In a preferred embodiment of the present invention, the preparation method of the compound shown in formula I, R¹ is hydrogen, C₁₋₆ alkyl, C₁₋₁₀ alkyl acyl, aryl acyl or (C₁₋₁₀ alkyl or aryl)₃ silyl; R² is C₁₋₁₀ alkyl acyl, aryl acyl; M is MgCl or MgBr.

In a more preferred embodiment of the present invention, the preparation method of the compound shown in formula I, R¹ is C₁₋₁₀ alkyl acyl, any acyl; R² is C₁₋₁₀ alkyl acyl, aryl acyl; M is MgCl or MgBr.

In a more preferred embodiment of the present invention, the preparation method of the compound shown in formula I, R¹ is acetyl; R² is acetyl or propionyl, M is MgBr.

In a more preferred embodiment of the present invention, the method for preparing fulvestrant as shown in formula I, comprises the following steps,
1) The compound shown in formula VIa and the compound shown in formula VIIa undergo Grignard addition reaction to obtain the compound shown in formula Va;
2) The compound shown in formula Va undergoes aromatization reaction and phenolic hydroxyl protection reaction with acetic anhydride to obtain the compound shown in formula IIIa;
3) The compound shown in formula IIIa and the compound shown in formula IV undergo substitution reactions to obtain the compound shown in formula II;
4) The compound shown in formula II undergoes oxidation reactions to obtain the compounds shown in formula I.

In a further preferred embodiment of the present invention, the method for preparing fulvestrant as shown in formula I, comprises the following steps.
1) The compound shown in formula VIa and the compound shown in formula VIIa undergo Grignard addition under the action of CuCl to obtain the compound shown in formula Va;
2) The compound shown in formula Va undergoes aromatization reaction under the action of CuBr₂ and LiBr, and the phenolic hydroxyl protection reaction with acetic anhydride to obtain the compound shown in formula IIIa;
3) The compound shown in formula IIIa and the compound shown in formula IV undergo substitution reaction under the action of sodium hydroxide to obtain the compound as shown in formula II;
4) The compounds shown in formula II undergo oxidation reactions under the action of hydrogen peroxide and peracetic acid to obtain the compounds shown in formula I;

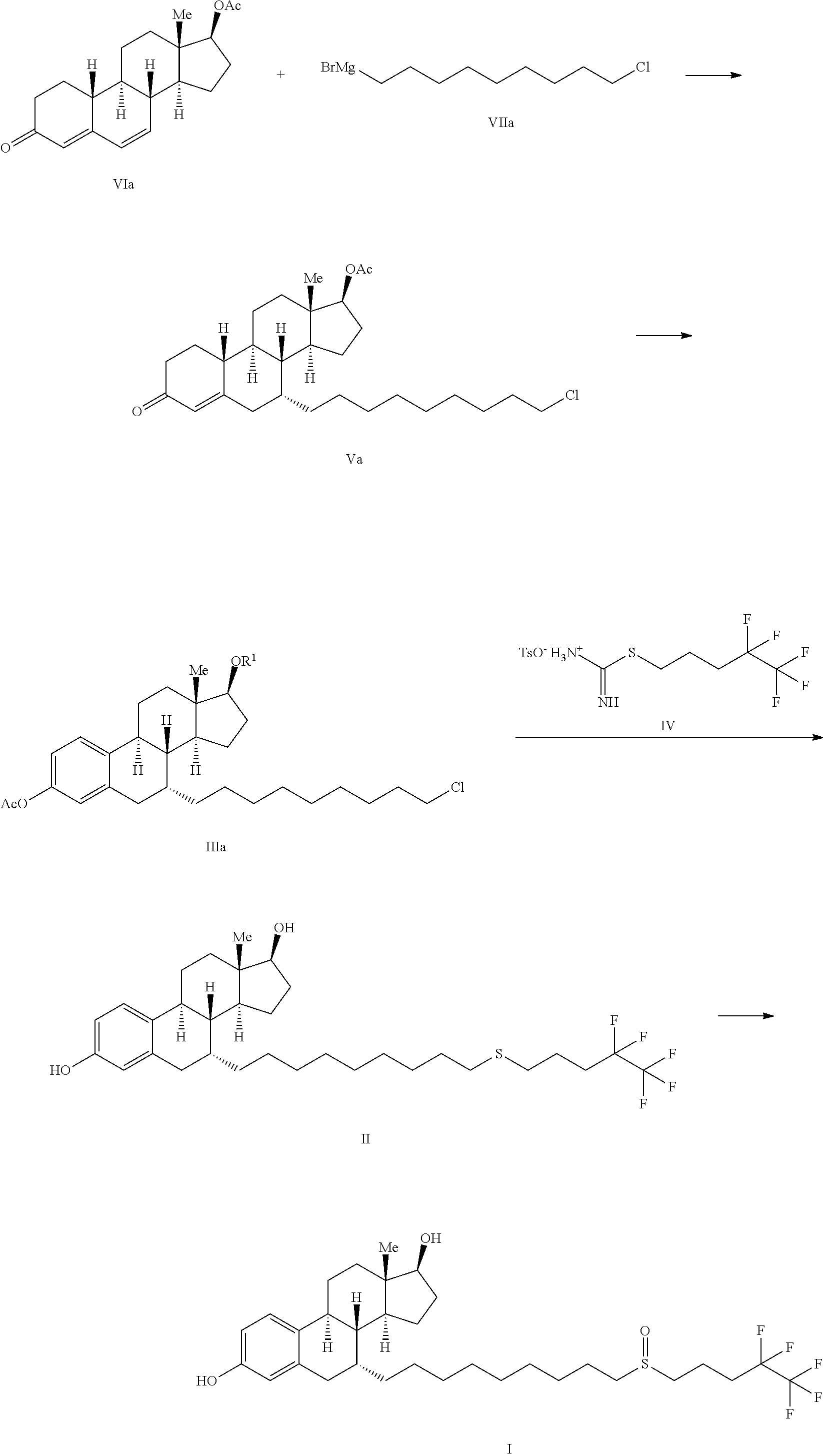

On the other hand, the present invention also provides method for preparing fulvestrant, comprising steps using 1,9-dichlorononane, 1-chloro-9-bromononane or 1-chloro-9-iodononane for the preparation of fulvestrant.

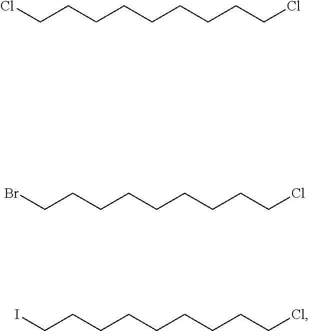

wherein, 1,9-dichlorononane or 1-chloro-9-bromononane is commercially available;
1-Chloro-9-bromononane can also be prepared by the chlorination of 9-bromo-1-nonanol.
1-Chloro-9-iodononane can be prepared according to: J. Org. Chem. 1963, 28, 1254-1259.

In addition, compound IV is commercially available or can be prepared according to WO2008044033.

Synthesis of compound VI can be referenced in: Canadian Journal of Chemistry 1984, 62, 2740-2747. That is, nandrolone undergoes oxidation by tetrachlorobenzoquinone to obtain dehydronandrolone, and the protection of hydroxyl group gives compound VI;

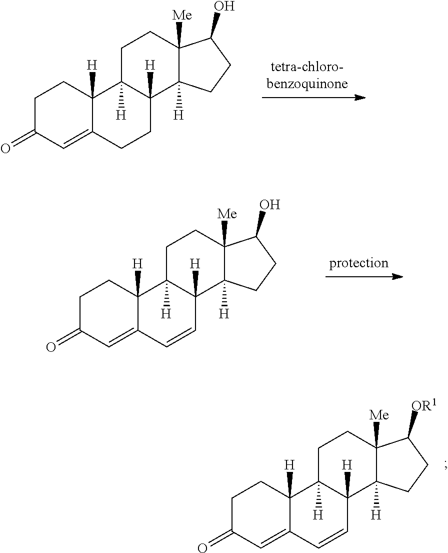

It can also be prepared by deprotection of dehydronandrolone acetate and replacing with other protective groups;

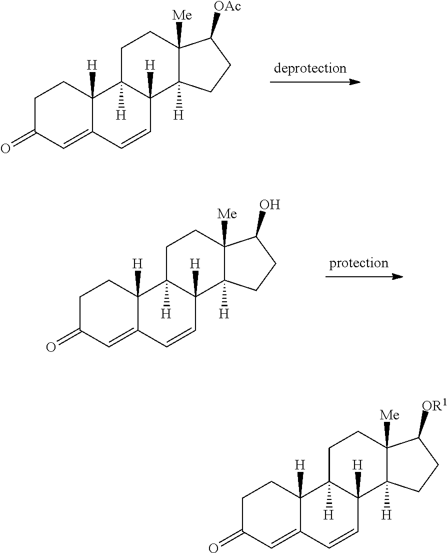

If commercially available, some of the products in the above steps can also be used to prepare the compounds shown in formula V in a shorter route; For example, the compound shown in formula V can be prepared by purchasing the aforementioned formula VI and then following the steps provided in the above method.

On the other hand, the present invention also provides a method for preparing fulvestrant, the method prepares the compound shown in formula V or formula III according to the method provided earlier in the present invention, and then prepares fulvestrant according to the known method from compound shown in formula V or III, which can be referred to the literatures: WO2002032922: WO2006015081; Organic Process Research & Development 2010, 14, 544-552.

The benefits of the present invention are:
1) The present invention uses a chlorine-containing metal reagent VII (in particular, a Grignard reagent VIIA containing chlorine at terminal position) for addition reaction, and the obtained product V can directly introduce pentafluoropentanethiol fragments with chlorine as the leaving group after aromatization, thereby avoiding the use of protective groups and the conversion of functional groups in the prior art, and shortening the steps of the synthesis route of fulvestrant.
2) The present invention uses the Grignard reagent VIIA containing chlorine at terminal position for the addition reaction with high selectivity, and the diastereomeric ratio can reach 9:1.
3) The conditions of the present invention are mild, and there are no reaction conditions for ultra-low temperature (e.g., less than −40° C.) and ultra-high temperature (e.g., greater than 80
4) The synthesis operation process of the present invention is simple, and qualified products can be obtained without column chromatography, which is easy to achieve industrial scale-up production.
5) The efficiency of fulvestrant synthesis of the present invention has a significant advantage.

Unless otherwise stated to the contrary, the terms used in the specification and claims have the following definitions:

The hydroxyl protecting group of the present invention is a suitable hydroxyl protection group known in this field, see hydroxyl protecting groups in the literature (“Protective Groups in Organic Synthesis”, 5Th Ed T. W. Greene & P. G. M. Wuts). As an example, the hydroxyl protection group can be (C₁₋₁₀ alkyl or aryl)₃ silanes, such as: triethylsilyl, triisopropylsilyl, tert-butyl dimethylsilyl, tert-butyl diphenylsilyl, etc.; it can be C₁₋₁₀ alkyl or substituted alkyl, such as: methyl, tert-butyl, allyl, benzyl, methoxymethyl, ethoxyethyl, 2-tetrahydropyranyl (THP), etc.; it can be (C₁₋₁₀ alkyl or aromatic) acyl, such as formyl, acetyl, benzoyl, p-toluyl benzoyl, etc.; it can be (C₁₋₆ alkyl or C₆₋₁₀ aryl) sulfonyl, it can also be (C₁₋₆ alkoxy or C₆₋₁₀ aryloxy) carbonyl.

The term “alkyl” refers to a saturated aliphatic hydrocarbon group, including linear or branched groups of 1 to 10 carbon atoms, preferably 1 to 6 carbon atoms. Non-limiting examples include, but are not limited to, methyl, ethyl, n-propyl, isopropyl, n-butyl, isobutyl, tert-butyl, sec-butyl, n-pentyl, 1,1-dimethylpropyl, 1,2-dimethylpropyl, 2,2-dimethylpropyl, 1-ethylpropyl, 2-methylbutyl, 3-methylbutyl, n-hexyl, 1-ethyl-2-methylpropyl, 1,1,2-trimethylpropyl, 1,1-dimethylbutyl, 1,2-dimethylbutyl, 2,2-dimethylbutyl, 1,3-dimethylbutyl, 2-ethylbutyl, 2-methylpentyl, 3-methylpentyl, 4-methylpentyl, 2,3-dimethylbutyl, and various isomers thereof, and the like. The alkyl can be substituted or unsubstituted and can be substituted at any available junction, and the substituent is preferably one or more than one groups, independently selected from alkyl, alkenyl, alkynyl, alkoxy, alkylmercapto, alkylamino, halogen, mercapto, hydroxyl, nitro, cyano, cycloalkyl, heterocycloalkyl, aryl, heteroaryl, cycloalkyloxy, heterocycloalkyloxy, cycloalkylmercapto, heterocycloalkylmercapto or oxo; Non-limiting examples include, but are not limited to, methyloxymethyl, ethyloxymethyl, benzyl, p-methoxybenzyl, p-methylbenzyl, 2-tetrahydropyranyl, and the like.

The term “aryl” refers to a 6 to 14-member all-carbon monocyclic or fused polycyclic (i.e., sharing adjacent carbon atoms) group of conjugated n electronic system, preferably 6 to 10 carbon atoms, more preferably phenyl and naphthyl, further preferably phenyl. The aryl can be substituted or unsubstituted, and the substituent is preferably one or more of the following groups, independently selected from alkyl, alkenyl, alkynyls, alkoxy, alkylmercapto, alkyl amino, halogen, mercapto, hydroxyl, nitro, cyano, cycloalkyl, heterocycloalkyl, aryl, heteroaryl, cycloalkyloxy, heterocycloalkyloxy, cycloalkylmercapto, or heterocycloalkymercapto.

Abbreviations:

| Abbreviation | Full name |
|---|---|
| MTBE | Methyl tert-butyl ether |
| Ac | Acetyl |
| TBS | tert-Butyldimethylsilyl |
| Bz | Benzoyl |
| Bn | Benzyl |
| MOM | Methoxymethyl |
| THP | 2-Tetrahydropyranyl |
| TsO⁻ | p-Toluenesulfonate |
| HPLC | High-performance liquid chromatography |

The following table contains the structures of the compounds involved in the examples

| Compound | Structure |
|---|---|
| I | 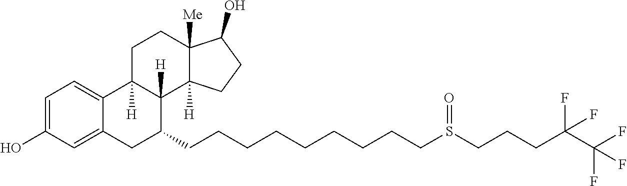 |
| II | 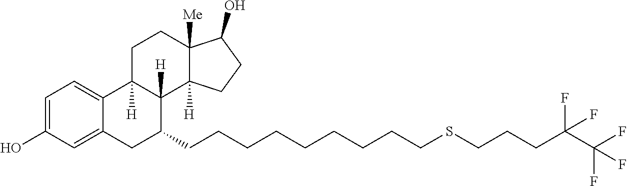 |
| IIIa | 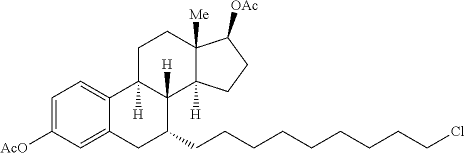 |
| IIIb | 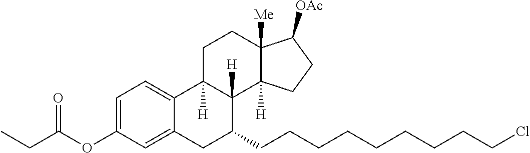 |
| IV | 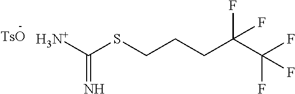 [SMILES: N=C([NH3+])SCCCC(F)(F)C(F)(F)F] |
| Va | 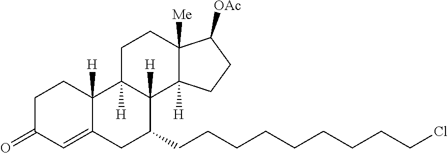 |
| Vb | 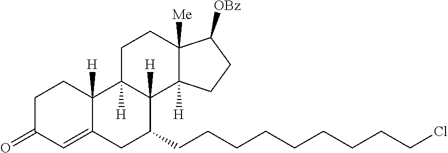 |
| Vc | 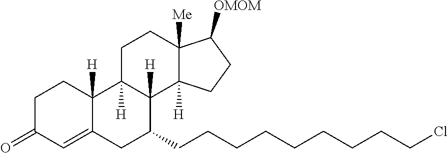 |
| Vd | 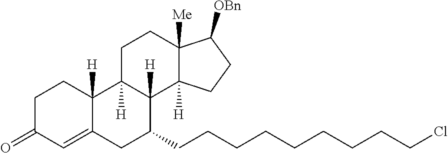 |
| Ve | 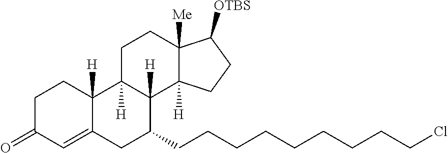 |
| Vf | 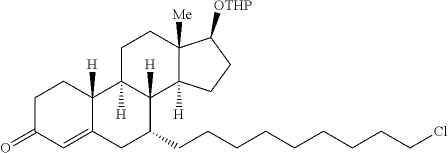 |
| Vg | 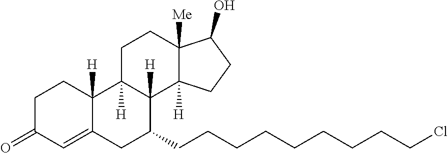 |
| VIa | 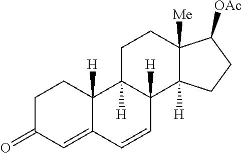 |
| VIb | 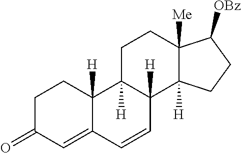 |
| VIc | 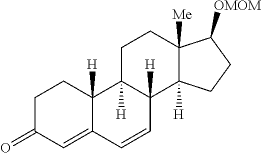 |
| VId | 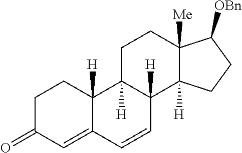 |
| VIe | 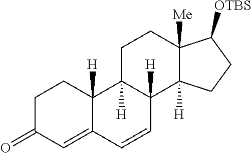 |
| VIf | 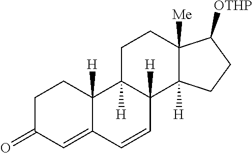 |
| VIg | 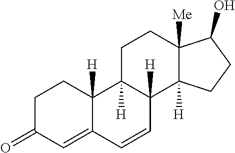 |
| VIIa | 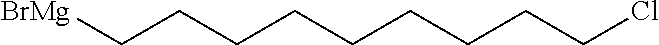 |
| VIIb | 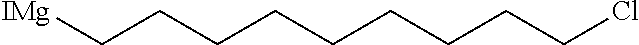 |
| VIIc | 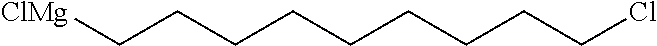 |
| VIId | 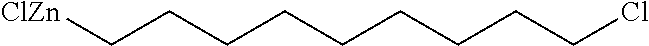 |
| VIIe | 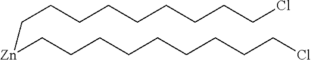 |
| VIIf | 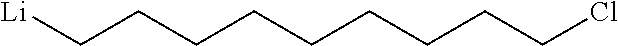 |
| VIIg | 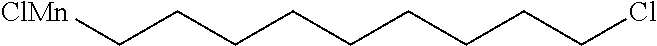 |
| VIIIa | 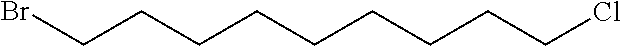 [SMILES: ClCCCCCCCCCBr] |
| VIIIb | 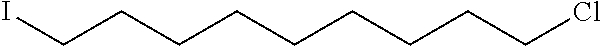 [SMILES: ClCCCCCCCCCI] |
| VIIIc | 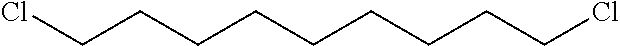 [SMILES: ClCCCCCCCCCCl] |
| IXa | 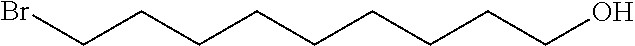 [SMILES: OCCCCCCCCCBr] |

### DETAILED DESCRIPTION OF THE EMBODIMENTS

The following examples illustrate the present invention, so that the professional and technical personnel could understand the present invention more fully. The examples are only used to illustrate the technical solution of the present invention, and do not limit the present invention in any way.

### Example 1: Preparation of Compound VIIIa

To a solution of compound IXa (50 g) in SOCl₂ (50 mL) was added pyridine (10 mL) dropwisely below 20° C. The reaction mixture was kept at 20° C. for 48 h. TLC showed that the reaction was complete, the mixture was quenched with cold water, and extracted with MTBE. The combined organic layer was washed with saturated sodium bicarbonate, dried, and concentrated to give compound VIIIa (52.6 g, 97% yield).

¹H NMR (400 MHz, Chloroform-d) δ 3.53 (td, J=6.7, 0.9 Hz, 2H), 3.41 (td, J=6.9, 0.8 Hz, 2H), 1.85 (p, J=7.0 Hz, 2H), 1.81-1.72 (m, 2H), 1.43 (dq, J=12.9, 6.8 Hz, 4H), 1.32 (d. J=4.5 Hz, 6H).

### Example 2: Preparation of Compound VIIa

To anhydrous THE (380 mL) was added magnesium turnings (6.42 g). Compound VIIIa (46.1 g) was added slowly at 20° C. and the reaction mixture was kept at room temperature for 2 h to obtain compound VIIa.

### Example 3: Preparation of Compound VIIa

To anhydrous THE (380 mL) was added compound VIIIa (46.1 g). Isopropylmagnesium bromide in THE (1.0 eq) was added dropwisely at 0° C. and the reaction mixture was stirred for 2 h to obtain compound VIIa.

### Example 4: Preparation of Compound VIIb

To anhydrous THF (380 mL) was added magnesium turnings (6 g). Compound VIIIb (48 g) was added slowly at 20° C. and the reaction mixture was kept at room temperature for 2 h to obtain compound VIIb.

### Example 5: Preparation of Compound VIIc

To anhydrous THF (380 mL) was added magnesium turnings (6 g). Compound VIIIc (32.7 g) was added slowly at 20° C. and the reaction mixture was kept at room temperature for 2 h to obtain compound VIIc.

### Example 6: Preparation of Compound VIId

To anhydrous THF (380 mL) was added magnesium turnings (6.42 g). Compound VIIIa (46.1 g) was added slowly at 20° C. and the reaction mixture was kept at room temperature for 2 h to obtain compound VIIa. The solution of compound VIIa was cooled to 0° C., and anhydrous ZnCl₂ in THF solution (1.0 eq) was added. The reaction mixture was stirred for 2 h, obtain compound VIId.

### Example 7: Preparation of Compound Vile

To anhydrous THF (380 mL) was added compound VIIIa (46.1 g). Diethylzinc in THF (0.5 eq) was added dropwisely at −30° C. and the reaction mixture was stirred for 2 h to obtain compound VIIe.

### Example 8: Preparation of Compound VIIf

To anhydrous THF (380 mL) was added compound VIIIa (46.1 g) tert-Butyllithium in THF (2.5 eq) was added dropwisely at −30° C. and the reaction mixture was stirred for 2 h to obtain compound VIIf.

### Example 9: Preparation of Compound VIIg

To anhydrous THF (380 mL) was added magnesium turnings (6.42 g). Compound VIIIa (46.1 g) was added slowly at 20° C. and the reaction mixture was kept at room temperature for 2 h to obtain compound VIIa. The solution of compound VIIa was cooled to 0° C., and anhydrous MnCl₂ in THF solution (1.0 eq) was added. The reaction mixture was stirred for 2 h, obtain compound VIIg.

### Example 10: Preparation of Compound Va

To THF (120 mL) was added compound VIa (20 g) and CuCl (1.42 g). Compound VIIa (1.3 eq) in THF was added slowly at −20° C. and the reaction mixture was kept stirring at −20° C. for 2 h. HPLC showed that the reaction was complete, the mixture was quenched with acetic acid, diluted with water and extracted with ethyl acetate. The combined organic layer was washed with saturated sodium bicarbonate, dried, and concentrated to give compound Va (28.0 g, 92% yield). The diastereomeric ratio of 7:70 is 9:1.

MS (ESI) m/z: 477 (M+H+).

¹H NMR (400 MHz, Chloroform-d) δ 5.82 (s, 1H), 4.61 (t, 1H), 3.52 (t, 2H), 2.52 (dd, J=14 Hz, J=2 Hz, 1H), 2.38˜2.42 (m, 1H), 2.21˜2.29 (m, 3H), 2.15˜2.21 (m, 1H), 2.03 (s, 3H), 1.96˜2.02 (m, 1H), 1.85˜1.88 (m, 1H), 1.68˜1.79 (m, 5H), 1.46˜1.60 (in, 4H), 0.95˜1.42 (m, 18H).

### Example 11: Preparation of Compound Va

To THF (120 mL) was added compound VIa (20 g) and CuCl (1.42 g). Compound VIIb (1.3 eq) in THF was added slowly at −20° C. and the reaction mixture was kept stirring at −20° C. for 2 h. HPLC showed that the reaction was complete, the mixture was quenched with acetic acid, diluted with water and extracted with ethyl acetate. The combined organic layer was washed with saturated sodium bicarbonate, dried, and concentrated to give compound Va (25.2 g, 83% yield). The diastereomeric ratio of 7α:7β is 8:1.

MS (ESI) m/z: 477 (M+H⁺).

¹H NMR (400 MHz, Chloroform-d) δ 5.82 (s, 1H), 4.61 (t, 1H), 3.52 (t, 2H), 2.52 (dd, J=14 Hz, J=2 Hz, 1H), 2 38˜2.42 (m, 1H), 2.21˜2 29 (m, 3H), 2.15˜2.21 (m, 1H), 2.03 (s, 3H), 1.96˜2.02 (m, 1-H), 1.85˜1.88 (m, 1H), 1.68˜4.79 (m, 5H), 1.46˜1.60 (m, 4H), 0.95˜1.42 (m, 18H).

### Example 12 Preparation of Compound Va

To THF (120 mL) was added compound VIa (20 g) and CuCl (1.42 g). Compound VIIc (1.3 eq) in THF was added slowly at −10° C. and the reaction mixture was kept stirring at −10° C. for 2 h. HPLC showed that the reaction was complete, the mixture was quenched with acetic acid, diluted with water and extracted with ethyl acetate. The combined organic layer was washed with saturated sodium bicarbonate, dried, and concentrated to give compound Va (22.7 g, 75% yield). The diastereomeric ratio of 7α:7β is 7:1.

MS (ESI) m/z: 477 (M+H⁺).

¹H NMR (400 MHz, Chloroform-d) δ 5.82 (s, 1H), 4.61 (t, 1H), 3.52 (t, 2H), 2.52 (dd, 0.1=14 Hz, J=2 Hz, 1H), 2.38˜2.42 (m, 1H), 2.21˜2.29 (m, 3H), 2.15˜2.21 (m, 1H), 2.03 (s, 3H), 1.96˜2.02 (m, 1H), 1.85˜1.88 (m, 1H), 1.68˜1.79 (m, 5H), 1.46˜1.60 (m, 4H), 0.95˜1.42 (m, 18H).

### Example 13: Preparation of Compound Va

To THF (120 mL) was added compound VIa (20 g) and CuCl (1.42 g). Compound VIId (1.3 eq) in THE was added slowly at −10° C. and the reaction mixture was kept stirring at −10° C. for 2 h. HPLC showed that the reaction was complete, the mixture was quenched with acetic acid, diluted with water and extracted with ethyl acetate. The combined organic layer was washed with saturated sodium bicarbonate, dried, and concentrated to give compound Va (11.0 g, 38% yield).

### Example 14: Preparation of Compound Va

To THF (120 mL) was added compound VIa (20 g) and CuCl (1.42 g). Compound VIIe (1.3 eq) in THF was added slowly at −10° C. and the reaction mixture was kept stirring at −10° C. for 2 h. HPLC showed that the reaction was complete, the mixture was quenched with acetic acid, diluted with water and extracted with ethyl acetate. The combined organic layer was washed with saturated sodium bicarbonate, dried, and concentrated to give compound Va (10.0 g, 33% yield).

### Example 15: Preparation of Compound Va

To THF (120 mL) was added compound VIa (20 g) and CuCl (1.42 g). Compound VIIf (1.3 eq) in THF was added slowly at −10° C. and the reaction mixture was kept stirring at −10° C. for 2 h. HPLC showed that the reaction was complete, the mixture was quenched with acetic acid, diluted with water and extracted with ethyl acetate. The combined organic layer was washed with saturated sodium bicarbonate, dried, and concentrated to give compound Va (8.0 g, 26% yield).

### Example 16: Preparation of Compound Va

To THF (120 mL) was added compound VIa (20 g) and CuCl (1.42 g). Compound VIIg (1.3 eq) in THF was added slowly at −10° C. and the reaction mixture was kept stirring at −10° C. for 2 h. HPLC showed that the reaction was complete, the mixture was quenched with acetic acid, diluted with water and extracted with ethyl acetate. The combined organic layer was washed with saturated sodium bicarbonate, dried, and concentrated to give compound Va (18.0 g, 60% yield).

### Example 17: Preparation of Compound Vb

To THF (15 mL) was added compound VIb (2 g) and CuCl (263 mg). Compound VIIa (1.5 eq) in THF was added slowly at −30° C. and the reaction mixture was kept stirring at −30° C. for 2 h. HPLC showed that the reaction was complete, the mixture was quenched with acetic acid, diluted with water and extracted with ethyl acetate. The combined organic layer was washed with saturated sodium bicarbonate, dried, and concentrated to give compound Vb (2.24 g, 78% yield). The diastereomeric ratio of 7α:7β is 9:1.

MS (ESI) m/z: 539 (M+H⁻).

### Example 18: Preparation of Compound Vc

To THF (15 mL) was added compound VIc (2 g) and CuBr (280 mg). Compound VIIa (1.5 eq) in THF was added slowly at 0° C. and the reaction mixture was kept stirring at 0° C. for 1 h. HPLC showed that the reaction was complete, the mixture was quenched with acetic acid, diluted with water and extracted with ethyl acetate. The combined organic layer was washed with saturated sodium bicarbonate, dried, and concentrated to give compound Vc (2.31 g, 70% yield). The diastereomeric ratio of 7α:7β is 5:1.

MS (ESI) m/z: 479 (M+H⁺).

### Example 19: Preparation of Compound Vd

To THF (15 mL) was added compound VId (2 g) and CuI (306 mg). Compound VIIa (1.2 eq) in THF was added slowly at −10° C. and the reaction mixture was kept stirring at −10° C. for 2 h. HPLC showed that the reaction was complete, the mixture was quenched with acetic acid, diluted with water and extracted with ethyl acetate. The combined organic layer was washed with saturated sodium bicarbonate, dried, and concentrated to give compound Vd (2.35 g, 81% yield). The diastereomeric ratio of 7α:7β is 8:1.

MS (ESI) m/z: 525 (M+H⁺).

### Example 20: Preparation of Compound Ve

To 2-methyltetrahydrofuran (15 mL) was added compound VIe (2 g) and CuOTf (244 mg). Compound VIIb (1.3 eq) in THF was added slowly at −30° C. and the reaction mixture was kept stirring at −30° C. for 2 h. HPLC showed that the reaction was complete, the mixture was quenched with acetic acid, diluted with water and extracted with ethyl acetate. The combined organic layer was washed with saturated sodium bicarbonate, dried, and concentrated to give compound Ve (2.28 g, 80% yield). The diastereomeric ratio of 7α:7β is 9:1.

MS (ESI) m/z: 549 (M+H⁻).

### Example 21: Preparation of Compound Vf

To THF (15 mL) was added compound VIf (2 g) and CuCl (122 mg). Compound VIIb (1.3 eq) in THF was added slowly at −20° C. and the reaction mixture was kept stirring at −20° C. for 2 h. HPLC showed that the reaction was complete, the mixture was quenched with acetic acid, diluted with water and extracted with ethyl acetate. The combined organic layer was washed with saturated sodium bicarbonate, dried, and concentrated to give compound Vf (2.25 g, 77% yield). The diastereomeric ratio of 7α:7β is 9:1.

MS (ESI) m/z: 519 (M+H⁺).

### Example 22: Preparation of Compound Vg

To THF (120 mL) was added compound VIg (20 g) and CuCl (1.42 g). Compound VIIa (2.5 eq) in THF was added slowly at −10° C. and the reaction mixture was kept stirring at −10° C. for 2 h. HPLC showed that the reaction was complete, the mixture was quenched with acetic acid, diluted with water and extracted with ethyl acetate. The combined organic layer was washed with saturated sodium bicarbonate, dried, and concentrated to give compound Vg (18.6 g, 58% yield). The diastereomeric ratio of 7α:7β is 8:1.

### Example 23: Preparation of Compound IIIa

To acetonitrile (15 mL) was added CuBr₂ (5.62 g). LiBr (1.55 g) and acetic anhydride (1.23 g). The reaction mixture was kept stirring at 20° C. for 0.5 h, and compound Va (5 g) in acetonitrile (15 mL) was added dropwisely. The reaction mixture was then kept stirring at 20° C. for 3 h. HPLC showed that the reaction was complete. The reaction mixture was quenched through dropwise addition into thiourea solution in toluene and water, and was kept stirring for 0.5 h. Then sodium bicarbonate was added and the reaction mixture was kept stirring for 10 min, filtered through celite, washed with toluene, and the combined organic layer was washed with saturated sodium chloride, dried, and concentrated to give compound IIIa (4.87 g, 90% yield).

MS (ESI) m/z: 517 (M+H⁺).

¹H NMR (400 MHz, Chloroform-d) δ 7.27 (d, J=9.6 Hz, 1H), 6.87 (dd, J=8.4 Hz, J=2.4 Hz, 1H), 6.78 (d, J=2.4 Hz, 1H), 4.70 (t, J=8.4 Hz, 1H), 3.53 (t, J=6.8 Hz, 2H), 2.90 (t, J=16.8 Hz, J=5.2 Hz, 1H), 2.75 (d, J=16.8 Hz, 1H), 2.29˜2.37 (m, 1H), 2.28 (s, 3H), 2.20˜2.26 (m, 1H), 2.06 (s, 3H), 1.84˜1.87 (m, 1H), 1.72˜1.79 (m, 3H), 1.59˜1.68 (m, 3H), 1.37˜1.55 (m, 8H), 1.18˜0.48 (m, 18H), 0.82 (s, 1H).

### Example 24: Preparation of Compound IIIb

To acetonitrile (10 mL) was added CuBr₂ (3.71 g), LiBr (1.02 g) and propionic anhydride (0.77 g). The reaction mixture was kept stirring at 20° C. for 0.5 h, and compound Va (3.3 g) in acetonitrile (10 mL) was added dropwisely. The reaction mixture was then kept stirring at 20° C. for 3 h, HPLC showed that the reaction was complete. The reaction mixture was quenched through dropwise addition into thiourea solution in toluene and water, and was kept stirring for 0.5 h. Then sodium bicarbonate was added and the reaction mixture was kept stirring for 10 min. filtered through celite, washed with toluene, and the combined organic layer was washed with saturated sodium chloride, dried, and concentrated to give compound IIIb (3.24 g, 88% yield).

MS (ESI) m/z: 531 (M+H⁺).

¹H NMR (400 MHz, Chloroform-d) δ 7.28 (d, J=8.8 Hz, 1H), 6.84 (dd, J=8.4 Hz, J=2.4 Hz, 1H), 6.78 (d, J=2.4 Hz, 1H), 4.67 (t, J=8.4 Hz, 1H), 3.52 (t, J=6.8 Hz, 2H), 2.90 (dd, J=16.8 Hz, J=5.2 Hz, 1H), 2.75 (d, J=16.8 Hz, 1H), 2.56 (q, J=15.2 Hz, 2H), 2.19˜2 39 (m, 3H), 2.06 (s, 3H), 1.83˜1.87 (m, 1H), 1.72˜1.79 (m, 3H), 1.62˜4.67 (m, 1H), 1.39˜1.58 (m, 8H), 1.17˜1.27 (m, 14H), 0.82 (s, 3H).

### Example 25: Preparation of Compound II

To DMF (25 mL) was added compound IIIa (4.38 g) and compound IV (4.5 g). 40% Sodium hydroxide solution (8.4 g) was added at 20° C. and the reaction mixture was kept stirring at 20° C. for 2 h HPLC showed that the reaction was complete. The reaction mixture was quenched with acetic acid, diluted with water and extracted with MTBE. The combined organic layer was washed with saturated sodium bicarbonate, saturated sodium chloride, dried, and concentrated to give compound II (4.51 g, 90% yield).

MS (ESI) m/z: 591 (M+H⁺).

¹H NMR (400 MHz, Chloroform-d) δ 7.15 (d, J=8.4 Hz, 1H), 6.63 (dd, J=8.8 Hz, J=2.4 Hz, 1H), 6.55 (d, J=2.4 Hz, 1H), 4.67 (s, 1H), 3.75 (t, J=7.6 Hz, 1H), 2.86 (dd, J=16.8 Hz, J=5.2 Hz, 1H), 2.71 (d, J=16.8 Hz, 1H), 2.59 (t, J=6 Hz, 2H), 2.50 (t, J=7.2 Hz, 2H), 2.28˜2.34 (m, 2H), 2.11˜2.23 (m, 3H), 1.84˜1.92 (m, 3H), 1.72˜1.75 (m, 1H), 1.53˜1.65 (m, 4H), 1.17˜1.50 (m, 24 J).

### Example 26: Preparation of Compound II

To DMF (20 mL) was added compound IIIb (3 g) and compound IV (3.08 g) 40% Sodium hydroxide solution (5.25 g) was added at 20° C. and the reaction mixture was kept stirring at 20° C. for 2 h HPLC showed that the reaction was complete. The reaction mixture was quenched with acetic acid, diluted with water and extracted with MTBE. The combined organic layer was washed with saturated sodium bicarbonate, saturated sodium chloride, dried, and concentrated to give compound II (2.77 g, 83% yield).

MS (ESI) m/z: 591 (M+H⁻).

¹H NMR (400 MHz, Chloroform-d) δ 7.15 (d, J=8.4 Hz, 1H), 6.63 (dd, J=8.8 Hz, J=2.4 Hz, 1H), 6.55 (d, J=2.4 Hz, 1H), 4.67 (s, 1H), 3.75 (t, J=7.6 Hz, 1H), 2.86 (dd, J=16.8 Hz, J=5.2 Hz, 1H), 2.71 (d, J=16.8 Hz, 1H), 2.59 (t, J=6 Hz, 2H), 2.50 (t, J=7.2 Hz, 2H), 2.28˜2.34 (m, 2H), 2.11˜2.23 (m, 3H), 1.84˜1.92 (m, 3H), 1.72˜1.75 (m, 1H), 1.53˜1.65 (m, 4H), 1.17˜1.50 (m, 24H).

### Example 27: Preparation of Compound I

To acetic acid (2.63 g) and 17.5% H₂O₂ (3.12 g) was added compound II (4.31 g) in ethyl acetate (15 mL). The reaction mixture was kept stirring at 20° C. for 10 h. HPLC showed that the reaction was complete. The reaction mixture was quenched with sodium sulfite solution and extracted with ethyl acetate. The combined organic layer was washed with saturated sodium bicarbonate, saturated sodium chloride, dried, and concentrated to give compound I (3.52 g, 80% yield, 99.7% purity).

MS (ESI) m/z: 607 (M+H⁺).

¹H NMR (400 MHz, Chloroform-d) δ 7.11 (d, J=8.4 Hz, H), 6.63 (d, J=8.4 Hz, 1H), 6.55 (s, 1H), 3.74 (t, J=8.5 Hz, 1H), 2.90-2.53 (m, 6H), 2.35-2.03 (m, 7H), 1.99-1.83 (m, 1H), 1.80-1.67 (m, 3H), 1.60 (qt, J=8.5, 3.3 Hz, 2H), 1.53-1.11 (m, 191), 1.08-0.95 (m, 1H), 0.77 (s, 3H).

Since this invention has been described in terms of specific embodiments thereof, certain modifications and equivalents will be apparent to those of ordinary skill in the art and are intended to be included within the scope of this invention.

We claim:

1. A compound as shown in formula V, 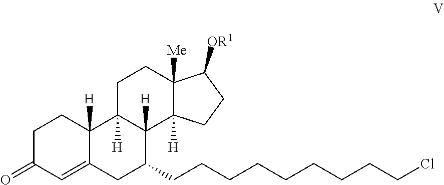
wherein:
R¹ is hydrogen or hydroxyl protecting groups.

2. The compound shown in formula V according to claim 1, wherein R¹ is hydrogen, benzyl, C₁₋₆ alkyl, C₁₋₁₀ alkyl acyl, aryl acyl or (C₁₋₁₀ alkyl or aryl)₃ silyl.

3. The compound shown in formula V according to claim 1, wherein R¹ is C₁₋₁₀ alkyl acyl or aryl acyl.

4. The compound shown in formula V according to claim 1, wherein R¹ is acetyl.

5. A preparation method of the compound shown in formula V according to claim 1, wherein it is prepared by an addition reaction of the compound shown in formula VI and the compound shown in formula VII, 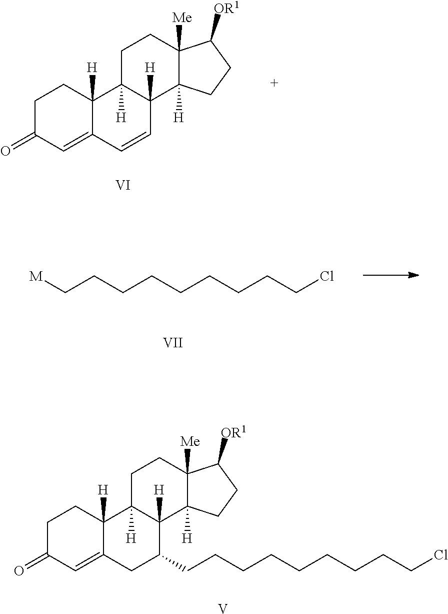
wherein:
R¹ s hydrogen or hydroxyl protecting groups.
M is MgCl, MgBr, MgI, Mg_{1/2}, ZnCl, ZnBr, ZnI, Zn_{1/2}, MnCl, Li or Na.

6. The preparation method of the compound shown in formula V according to claim 5, wherein R¹ is hydrogen, benzyl, C₁₋₆ alkyl, C₁₋₁₀ alkyl acyl, aryl acyl or (C₁₋₁₀ alkyl or aryl)₃ silyl; M is MgCl or MgBr.

7. The preparation method of the compound shown in formula V according to claim 5, wherein R¹ is C₁₋₁₀ alkyl acyl, aryl acryl; M is MgCl or MgBr.

8. The preparation method of the compound shown in formula V according to claim 5, wherein R¹ is acetyl.

9. A compound as shown in formula VII, 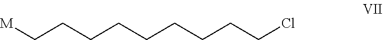
wherein M is MgCl, MgBr, MgI, Mg_{1/2}, ZnCl, ZnBr, ZnI, Zn_{1/2}, MnCl, Li or Na.

10. A method for preparing a compound as shown in formula VII, wherein it is prepared by reaction with metal or exchange reaction with metal from compound as described in formula VIII, 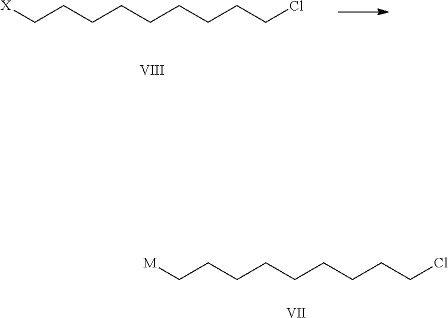
wherein X is Cl, Br or I, M is MgCl, MgBr, MgI, Mg_{1/2}, ZnCl, ZnBr, ZnI, Zn_{1/2}, MnCl, Li or Na.

11. The compound as shown in formula VII according to claim 9, wherein the compound shown in formula VIIA, 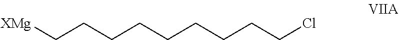
wherein X is Cl, Br or I.

12. A method for preparing a compound as shown in formula VIIA, wherein it is prepared by Grignard reaction from the compound as shown in formula VIII with magnesium metal, or with Grignard reagents through Grignard exchange reaction, 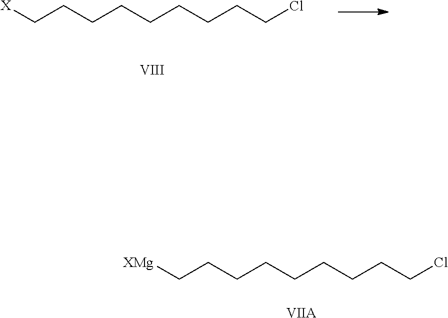
wherein X is Cl, Br or I.

13. A compound as shown in formula III, 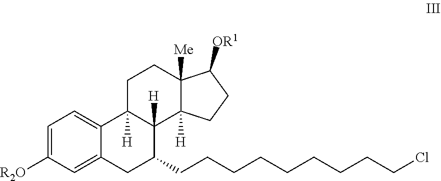
wherein R¹ is hydrogen or hydroxyl protecting groups; R² is hydroxyl protecting groups.

14. The compound shown in formula III according to claim 13, wherein R¹ is benzyl, C₁₋₆ alkyl, C₁₋₁₀ alkyl acyl, aryl acyl or (C₁₋₁₀ alkyl or aryl)₃ silyl; R² is C₁₋₁₀ alkyl acyl or aryl acyl.

15. The compound shown in formula III according to claim 13, wherein R¹ is acetyl; R² is acetyl or propionyl.

16. A preparation method for a compound as shown in formula III, wherein it is prepared by aromatization reaction and phenolic hydroxyl protection reaction of the compound shown in formula V, 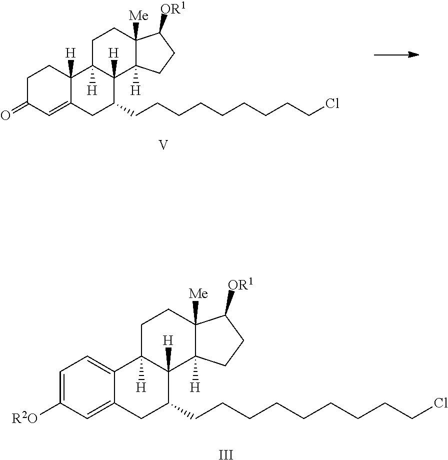
wherein R¹ is hydrogen or hydroxyl protecting groups; R¹ is hydroxyl protecting groups.

17. The method for preparing the compound shown in formula III according to claim 16, wherein R¹ is hydrogen, benzyl, C₁₋₆ alkyl, C₁₋₁₀ alkyl acyl, aryl acyl or (C₁₋₁₀ alkyl or aryl)₃ silyl; R² is C₁₋₁₀ alkyl acyl or aryl acyl.

18. The method for preparing the compound shown in formula III according to claim 16, wherein R¹ is C₁₋₁₀ alkyl acyl or aryl acyl; R² is C₁₋₁₀ alkyl acyl or aryl acyl.

19. The method for preparing the compound shown in formula III according to claim 16, wherein R¹ is acetyl; R² is acetyl or propionyl.

20. A method for preparing fulvestrant as shown in formula I, wherein it comprises the following steps,
1) The compound shown in formula VI and the compound shown in formula VII undergo addition reaction to obtain the compound shown in formula V;
2) The compound shown in formula V undergoes aromatization reaction and phenolic hydroxyl protection reaction to obtain the compound shown in formula III;
3) The compound shown in formula III and the compound shown in formula IV undergo substitution reaction to obtain the compound as shown in formula II;
4) The compound shown in formula II undergoes oxidation reaction to obtain the compound shown in formula I; 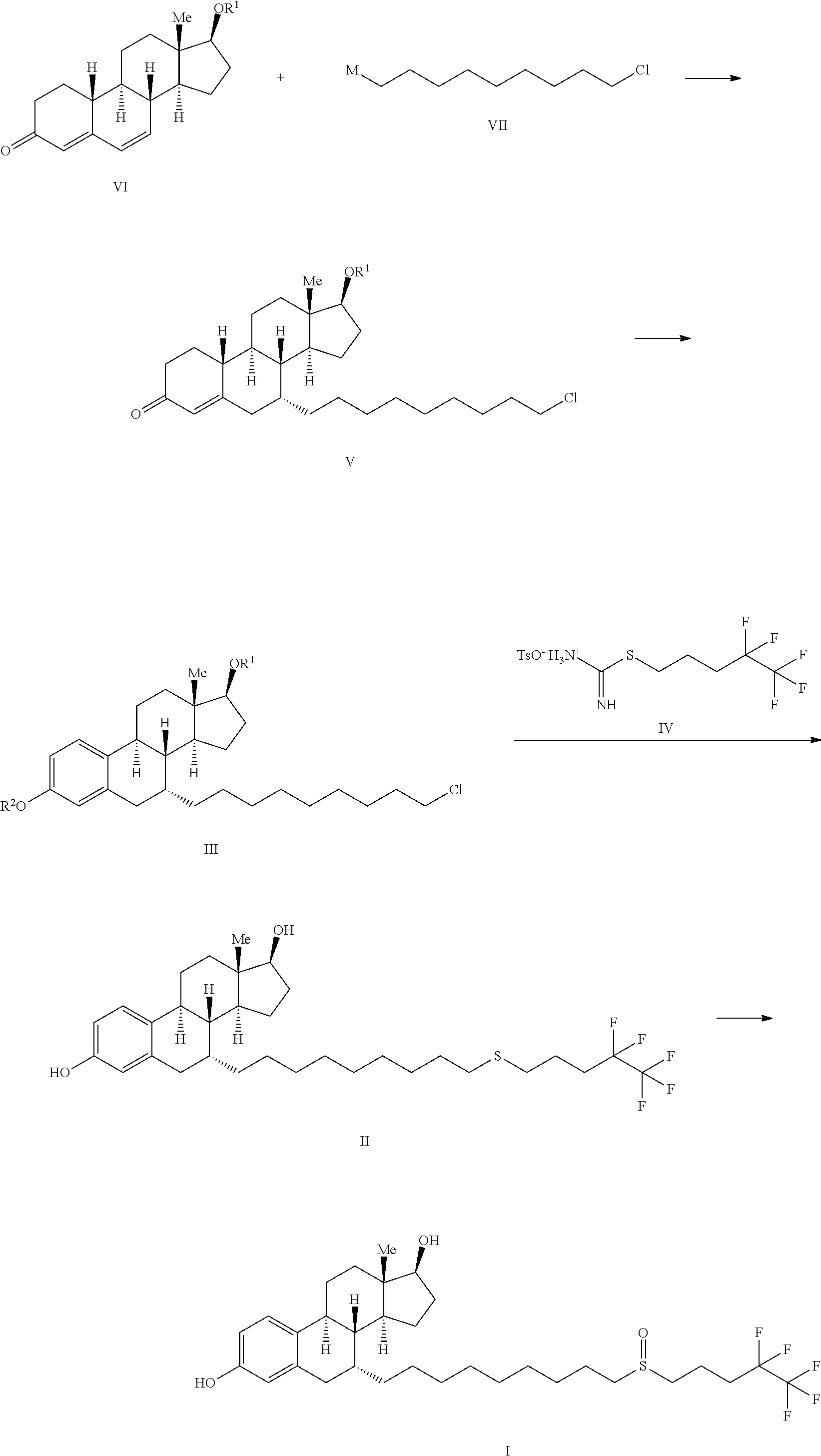
wherein R¹ is hydrogen or hydroxyl protecting groups; R² is hydroxyl protecting groups; M is MgCl, MgBr, MgI, Mg_{1/2}, ZnCl, ZnBr, ZnI, Zn_{1/2}, MnCl, Li or Na.

21. The method of preparing the compound shown in formula I according to claim 20, wherein R¹ is hydrogen, benzyl, C₁₋₆ alkyl, C₁₋₁₀ alkyl acyl, aryl acyl or (C₁₋₁₀ alkyl or aryl)₃ silyl; R² is C₁₋₁₀ alkyl acyl or aryl acyl; M is MgCl or MgBr.

22. The method of preparing the compound shown in formula I according to claim 20, wherein R¹ is C₁₋₁₀ alkyl acyl or aryl acyl; R² is C₁₋₁₀ alkyl acyl or aryl acyl; M is MgCl or MgBr.

23. The method of preparing the compound shown in formula I according to claim 20, wherein R¹ is acetyl; R² is acetyl or propionyl; M is MgBr.

24. A method for preparing fulvestrant as shown in formula I, wherein it comprises the following steps,
1) The compound shown in formula VIa and the compound shown in formula VIIa undergo addition reaction to obtain the compound shown in formula Va;
2) The compound shown in formula Va undergoes aromatization reaction and phenolic hydroxyl protection reaction to obtain the compound shown in formula IIIa;
3) The compound shown in formula IIIa and the compound shown in formula IVa undergo substitution reaction to obtain the compound as shown in formula II;
4) The compound shown in formula II undergoes oxidation reaction to obtain the compound shown in formula I.

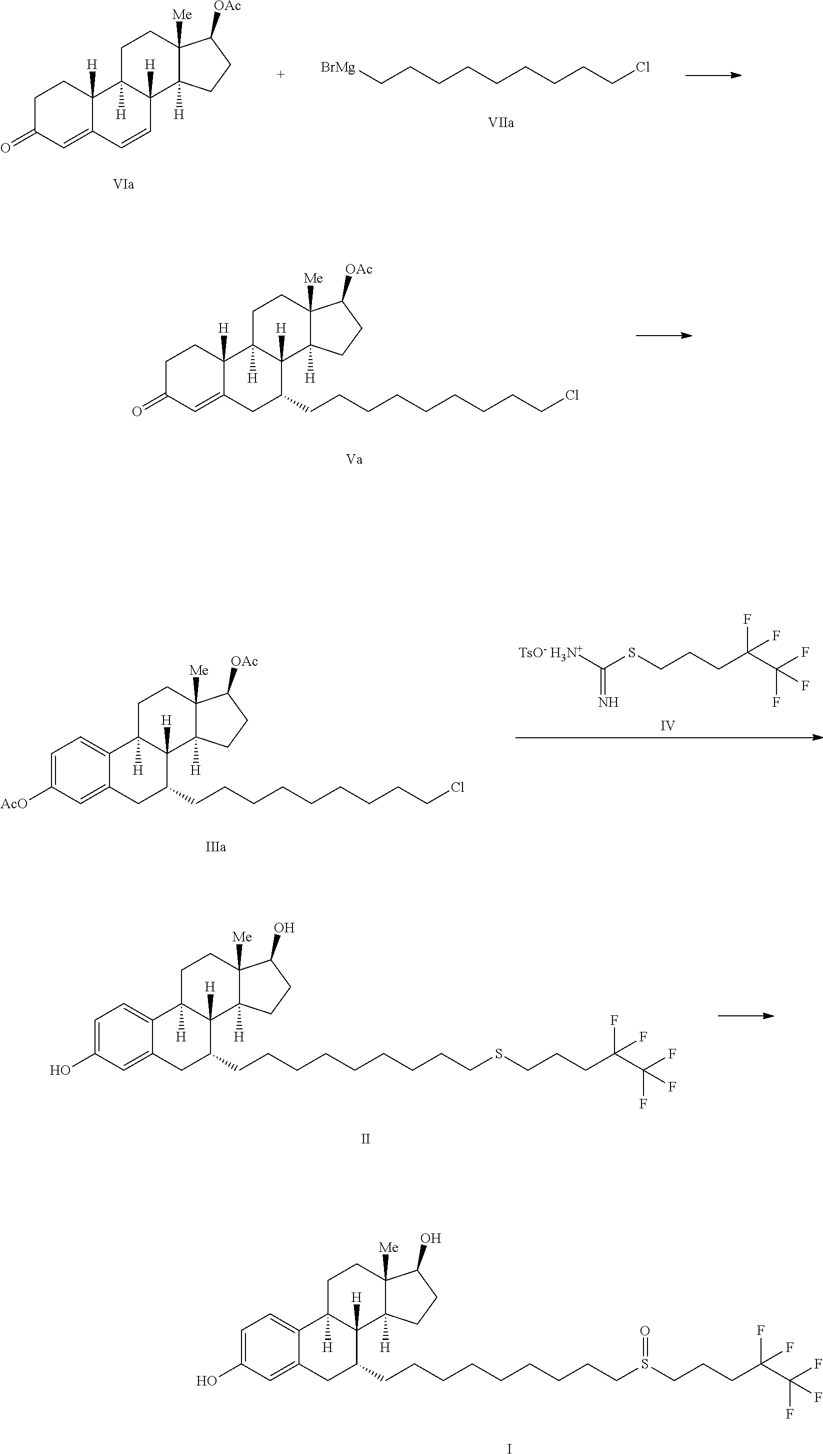

25. A method for preparing fulvestrant, wherein it comprises the method for preparing the compounds shown in formula VII, VIIA, V, III according to any one of claims 5-8, 10-12 or 16-19, or a steps for preparing fulvestrant by any one of the compounds shown in formula V according to any one of claims 1-4, the compounds shown in formula VII or VIIA according to claims 9-11, or the compounds shown in formula III according to any one of claims 13-15.

26. A method for preparing fulvestrant, wherein it comprises steps for preparing fulvestrant by 1,9-dichlorononane, 1-chloro-9-bromononane or 1-chloro-9-iodononane.
